(19) United States
(12) Patent Application Publication
(10) Pub. No.: US 2025/0344975 A1
(43) Pub. Date: Nov. 13, 2025

(54) METHOD AND APPARATUS FOR ENHANCING PREDICTION OF NEURODEVELOPMENTAL DISORDER USING FUNDUS IMAGE

(71) Applicant: Ellie Ye-Rin Sul, Seoul (KR)

(72) Inventor: Ellie Ye-Rin Sul, Seoul (KR)

(21) Appl. No.: 18/657,782

(22) Filed: May 8, 2024

(51) Int. Cl. A61B 5/16; A61B 3/00; A61B 3/12; A61B 5/00

(52) U.S. Cl. CPC A61B 5/168; A61B 3/0025; A61B 3/12; A61B 5/7267; A61B 5/7275; A61B 5/7485

(57) ABSTRACT

Provided are apparatuses, a non-transitory computer-readable medium or media, for enhancing prediction of neurodevelopmental disorder using a fundus image of a subject. In certain aspects, disclosed a method including the steps of: receiving the fundus image; segmenting a region of interest for the fundus image based on a machine learning model; mapping an adversarial noise to the region of interest; processing the fundus image to classify one or more features contained in the region of interest, which is mapped by the adversarial noise, using the machine learning model; and predicting, based on a classification, whether the fundus image is indicative of presence of neurodevelopmental disorder in the subject.

### A. TECHNICAL FIELD

The present disclosure relates to predicting neurodevelopmental disorders using a biometric image, more particularly, to an apparatus and method for enhancing prediction of the neurodevelopmental disorder using a fundus image based on a machine learning model.

### B. DESCRIPTION OF THE RELATED ART

With development of artificial intelligence learning models, many machine learning models are being used to read medical images. In a fundus image of the medical images, the machine learning models are currently being used to support an image reading, an image finding, an image diagnosis to predict a disease of a patient. More specifically, a method of supporting the image reading, the image finding, the image diagnosis of the fundus image is to obtain the fundus image from the patient, extract feature from the fundus image based on the machined learning models, provide the feature to a practitioner, and predict the patient's disease based on it. In this case, the feature includes various information for the fundus image.

Meanwhile, numerous children are suffering from neurodevelopmental disorders. In fact, about one in six children in the U.S. have one or more developmental disabilities or other developmental delays (CDC 2022). The early diagnosis of neurodevelopmental disorders is very important as it is the key to increasing the percentage of being cured of the disorders. There are many methods of diagnosing children with neurodevelopmental disorders, but the method using fundus image is very effective as it is a non-invasive method that can be used to diagnose children with neurodevelopmental disorders such as autism spectrum disorder (ASD) and attention-deficit/hyperactivity disorder (ADHD). And because this is a newly discovered method, there is a need for improvement with more research. There were a number of previous studies conducted on the diagnosis of various neurodevelopmental disorders using fundus images. For example, the research found that fundus images can be used to diagnose Parkinson's disease, ASD, etc. Although these researches brought very important findings, it needs to be improved through more research on how to better capture the important parts of a fundus image such as the optic nerve and blood vessels.

### SUMMARY OF THE DISCLOSURE

In one aspect of the present disclosure, an apparatus for enhancing prediction of neurodevelopmental disorder using a fundus image of a subject includes a processor; and a memory comprising one or more sequences of instructions which, when executed by the processor, causes steps to be performed comprising receiving the fundus image, segmenting a region of interest for the fundus image based on a machine learning model, mapping an adversarial noise to the region of interest, processing the fundus image to classify one or more features contained in the region of interest, which is mapped by the adversarial noise, using the machine learning model, and predicting, based on a classification, whether the fundus image is indicative of presence of neurodevelopmental disorder in the subject.

Desirably, the steps further may comprise generating images in which a specific area of the fundus image is enlarged, after receiving the fundus image.

In another aspect of the present disclosure, an apparatus for predicting of neurodevelopmental disorder using a fundus image of a subject includes a processor; and a memory comprising one or more sequences of instructions which, when executed by the processor, causes steps to be performed comprising receiving the fundus image, receiving the fundus image, segmenting a plurality of regions of interest for the fundus image based on a machine learning model, mapping an adversarial noise to each of the plurality of the regions of interest, processing the fundus image to classify one or more features contained in the each of the plurality of the regions of interest, which is mapped by the adversarial noise, using the machine learning model, obtaining prediction values for the each of the plurality of the regions of interest, based on a classification, whether the fundus image is indicative of presence of neurodevelopmental disorder in the subject, and comparing the prediction values and determining a difference between the prediction values.

Desirably, the adversarial noise mapping to each of the plurality of the regions of interest may have a same value.

Desirably, the region of interest may include at least one of a retinal vessel, optic cup, optic disc in the fundus image.

Desirably, the adversarial noise may include at least one of gradation levels of R, G, and B pixels of the fundus image, a color of R, G, and B pixels of the fundus image, and a contrast ratio of the fundus image.

Desirably, the neurodevelopmental disorder may include autism spectrum disorder or attention-deficit/hyperactivity disorder.

### BRIEF DESCRIPTION OF THE DRAWINGS

References will be made to embodiments of the disclosure, examples of which may be illustrated in the accompanying figures. These figures are intended to be illustrative, not limiting. Although the disclosure is generally described in the context of these embodiments, it should be understood that it is not intended to limit the scope of the disclosure to these particular embodiments.

FIG. 1 shows a flowchart of an illustrative process for generating a feature information of a fundus image by an apparatus according to embodiments of the present disclosure.

FIG. 2 is a schematic diagram of an illustrative apparatus for enhancing prediction of neurodevelopmental disorder using a fundus image according to embodiments of the present disclosure.

FIG. 3 is a schematic diagram of an illustrative system for predicting neurodevelopmental disorder using a fundus image according to embodiments of the present disclosure.

FIG. 4 is an exemplary diagram of a first architecture of a machine learning model for predicting of neurodevelopmental disorder according to embodiments of the present disclosure.

FIG. 5 is an exemplary diagram of a second architecture of a machine learning model for predicting of neurodevelopmental disorder according to embodiments of the present disclosure.

FIG. 6 is an exemplary diagram of a third architecture of a machine learning model for predicting of neurodevelopmental disorder according to embodiments of the present disclosure.

FIG. 7 is an exemplary diagram of a fourth architecture of a machine learning model for predicting of neurodevelopmental disorder according to embodiments of the present disclosure.

FIGS. 8A and 8B are a view showing an illustrative process for generating a pre-fundus image by apparatus according to embodiments of the present disclosure.

FIG. 9 shows a table of performance comparison of overall ASD classification results based on comparison with state-of the-art methods according to embodiments of the present disclosure.

FIG. 10 shows a confusion matrix of a proposed method according to embodiments of the present disclosure.

FIG. 11 shows a table of result of an ablation study based on various networks according to embodiments of the present disclosure.

FIG. 12 is an exemplary flow diagram showing a prediction process of neurodevelopmental disorder using a fundus image according to embodiments of the present disclosure.

### DETAILED DESCRIPTION OF PREFERRED EMBODIMENTS

In the following description, for purposes of explanation, specific details are set forth in order to provide an understanding of the disclosure. It will be apparent, however, to one skilled in the art that the disclosure can be practiced without these details. Furthermore, one skilled in the art will recognize that embodiments of the present disclosure, described below, may be implemented in a variety of ways, such as a process, an apparatus, a system, a device, or a method on a tangible computer-readable medium.

Components shown in diagrams are illustrative of exemplary embodiments of the disclosure and are meant to avoid obscuring the disclosure. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components that may be implemented in software, hardware, or a combination thereof.

It shall also be noted that the terms “coupled,” “connected,” “linked,” or “communicatively coupled” shall be understood to include direct connections, indirect connections through one or more intermediary devices, and wireless connections.

Furthermore, one skilled in the art shall recognize: (1) that certain steps may optionally be performed; (2) that steps may not be limited to the specific order set forth herein; and (3) that certain steps may be performed in different orders, including being done contemporaneously.

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” or “in embodiments” in various places in the specification are not necessarily all referring to the same embodiment or embodiments.

In the following description, it shall also be noted that the terms “learning” shall be understood not to intend mental action such as human educational activity because of referring to performing machine learning by a processing module such as a processor, a CPU, an application processor, micro-controller, so on.

An “image” is defined as a reproduction or imitation of the form of a person or thing, or specific characteristics thereof, in digital form. An image can be, but is not limited to, a JPEG image, a PNG image, a GIF image, a TIFF image, or any other digital image format known in the art. “Image” is used interchangeably with “photograph”.

A “feature(s)” or “feature information” is defined as a group of one or more descriptive characteristics of subjects that can discriminate for disease. A feature can be a numeric attribute.

The terms “comprise/include” used throughout the description and the claims and modifications thereof are not intended to exclude other technical features, additions, components, or operations.

The terminology used herein is for purposes of describing particular embodiments only and is not intended to be limiting. The defined terms are in addition to the technical and scientific meanings of the defined terms as commonly understood and accepted in the technical field of the present teachings.

Relative terms may be used to describe the various elements' relationships to one another, as illustrated in the accompanying drawings. These relative terms are intended to encompass different orientations of the device and/or elements in addition to the orientation depicted in the drawings.

Unless the context clearly indicates otherwise, the singular forms “a,” “an,” and “the” are intended to include the plural forms as well. Also, when description related to a known configuration or function is deemed to render the present disclosure ambiguous, the corresponding description is omitted.

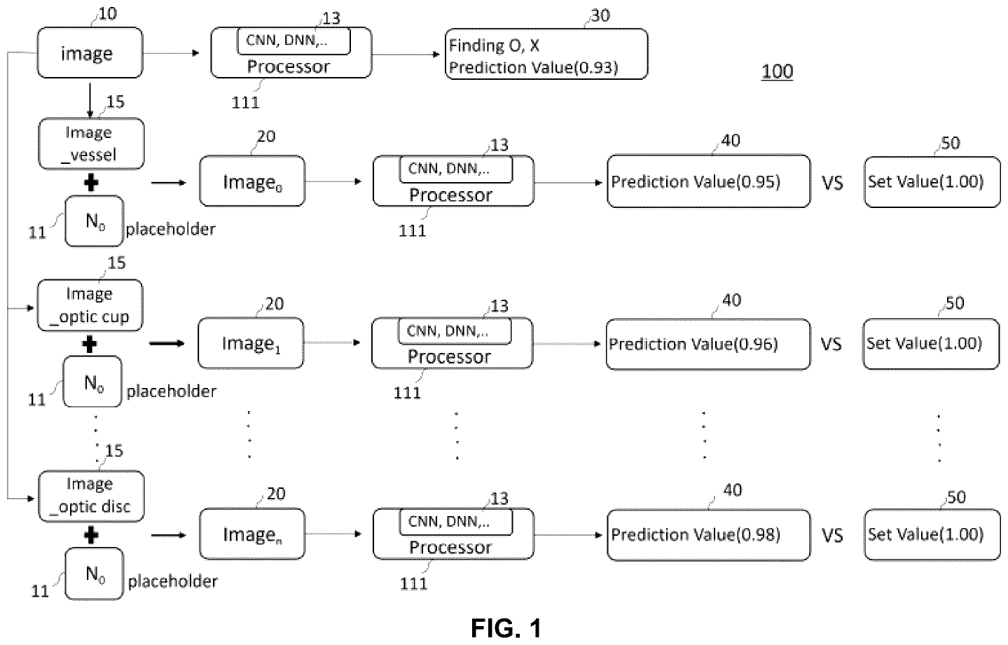

FIG. 1 shows a flowchart of an illustrative process for generating a feature information of a fundus image by an apparatus according to embodiments of the present disclosure. As depicted, in embodiments, a processor 111 may extract a first feature information 30 from a first fundus image 10 that is photographed by imaging means such as a camera, using a machine learning model 13 (e.g., CNN, DNN, etc.). The extracted first feature information 30 may be stored in a memory unit 113 or a storage device 115 described below. In embodiments, the machine learning model 13 may be installed into a processor 111 and executed by the processor 111. The machine learning model 13 may be installed into a computer-readable medium or media (not shown in FIG. 1) and executed by the computer-readable medium or media. In alternative embodiments, the machine learning model 13 may be installed into the memory unit 113 or the storage device 115 and executed by the processor 111.

In addition, the processor 111 may store clinical information of a subject (e.g., a patient) in the memory unit 113 or the storage device 115 in advance. In embodiments, the processor 111 may extract the first feature information of the fundus image 10 based on the machine learning model 13 by using the clinical information of the subject stored in the memory unit 113 or the storage device 115. In embodiments, the clinical information may be, but is not limited to, the age, sex, medical history, questionnaire information, test measurement values, exercise habits, eating habits, family history related to the medical history, alcohol consumption, smoking status. In embodiments, the questionnaire information may include neuromedical questionnaire that a practitioner (e.g., a medical doctor) can perform on the subject or may mean ideal findings currently observed to the subject, unlike medical history thereof.

In embodiments, the first feature information 30 may be various information that it can support an entity (e.g., a practitioner or a computing device) reading an fundus image such as predicting or diagnosing disease. For instance, when predicting or diagnosing autism spectrum disorder in the fundus image of the subject, the first feature information may include at least one of various information such as retinal vessel information, optic cup information, or optic disc information in the fundus image. In addition, the first feature information may include at least one of optic nerve vascular information that provides information on major organs of the eyeball, binocular classification information indicating whether the fundus image is an image of the left eye or the right eye, location information indicating a location of at least one of the macular and optic disk, and partitioning information indicating a segment of the fundus image and the like in the fundus image.

In this case, in embodiments, on the results performed by the processor 111, the apparatus 100 may appear the finding (Finding O, X) indicating the presence or absence of neurodevelopmental disorder in the fundus image on the basis of the first feature information, or a prediction value indicating the presence or absence of the finding on a display device 130, 330 describe below. In embodiments, the prediction value may be expressed as a percentage or a number between 0 and 1, an explanation of presence or absence of the finding and the prediction value will be described in more detail below.

Meanwhile, in embodiments, the processor 111 may generate second fundus images (Image_ᵥₑₛₛₑₗ, Image__{optic cup}, . . . , Image__{optic disc}) 15 by segmenting a region of interest (e.g., bio-marker) in the first fundus image 10 based on the machine learning model. The second fundus image 15 may be one of the fundus image with only vessel, the fundus image with only optic cup, and the fundus image with only optic disc isolated from the fundus image. In embodiments, the processor 111 may generate third fundus images (Image₀, Image₁ . . . , Imageₙ) 20, that are another image artificially made similar to the second fundus images 15 using a GAN (Generative Adversarial Networks) learning model, from the second fundus images 15.

In preferred embodiments, the processor 111 may generate the third fundus images 20 that is made using the second fundus images 15. The second fundus images can more accurately reflect its own feature information rather than another image artificially made similar to the second fundus images 15 using a GAN (Generative Adversarial Networks) learning model. Therefore, the feature information of the third fundus images 20 that are made using the second fundus images may have better reliability. More specifically, the processor 111 may map an adversarial factor 11 into the second fundus images 15. By doing so, the first feature information 30 of the first fundus image 10 is changed so that it may generate the third fundus images 20 including new feature information 40. In embodiments, the generated third fundus images 20 including the new feature information 40 may be stored in the memory unit 113 or the storage device 115 described below.

In embodiments, the adversarial factor 11 may be adversarial noises (N₀, . . . , Nₙ) that are attacked to the second fundus images (Image_ᵥₑₛₛₑₗ, Image__{optic cup}, Image__{optic disc}) 15. For example, the adversarial noise may include at least one of a value that adjusts the gradation level of the R, G, and B pixels representing the second fundus images 15, a value that adjusts the color of the R, G, and B pixels of the second fundus images 15, a value that locally adjusts the contrast ratio in the second fundus images 15. In embodiments, the adversarial noises (No) values may be all the same. It should be noted that the adversarial factor 11 may include any factor that can change the new feature information of the second fundus images 15.

In embodiments, the prediction value 40 of the third fundus images 20 may be obtained based on the machine learning model 13. The processor 111 may predict diseases such as autism in the fundus image by comparing the predicted value with the set value 50. Also, by comparing the predicted values for the third fundus images, the processor 111 may determine which third image has a significant influence on predicting the disease. Here, If the set value is 1, it may mean that the generated fundus image (the third fundus image 20) is a fundus image that is close to an abnormal fundus image in which any disease can be predicted or diagnosed thereon. In such case, it may mean that there is the finding. if the set value is 0, it may mean that the generated fundus image (the third fundus image 20) is a fundus image that is close to a normal fundus image in which any disease cannot be predicted or diagnosed thereon. In such case, it may mean that there is no the finding.

In embodiments, the apparatus 100 may compare the predicted values for the third fundus images 20 with each other by the processor 111 and display the differences between the predicted values on the display device 130 so that it may provide the difference to the entity described below.

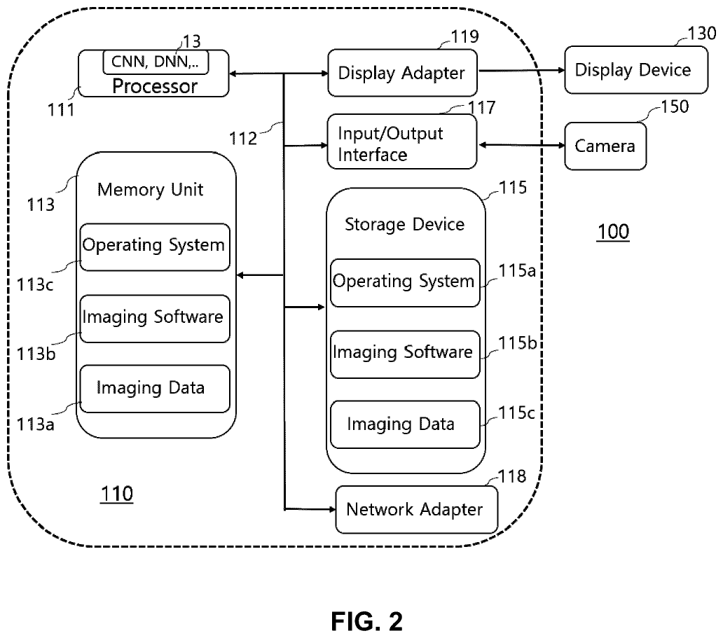

FIG. 2 is a schematic diagram of an illustrative apparatus for enhancing prediction of neurodevelopmental disorder using a fundus image according to embodiments of the present disclosure.

As depicted, the apparatus 100 may include a computing device 110, a display device 130 and a camera 150. In embodiments, the computing device 110 may include, but is not limited thereto, one or more processor 111, a memory unit 113, a storage device 115, an input/output interface 117, a network adapter 118, a display adapter 119, and a system bus 112 connecting various system components to the memory unit 113. In embodiments, the apparatus 100 may further include communication mechanisms as well as the system bus 112 for transferring information. In embodiments, the communication mechanisms or the system bus 112 may interconnect the processor 111, a computer-readable medium, a short range communication module (e.g., a Bluetooth, a NFC), the network adapter 118 including a network interface or mobile communication module, the display device 130 (e.g., a CRT, a LCD, etc.), an input device (e.g., a keyboard, a keypad, a virtual keyboard, a mouse, a trackball, a stylus, a touch sensing means, etc.) and/or subsystems. In embodiments, the camera 150 may include an image sensor (not shown) that captures an image of an subject and photoelectrically converts the image into an image signal, and may photograph a fundus image of the subject using the image sensor. The photographed fundus image may be stored in the memory unit 113 or the storage device 115, or may be provided to the processor 111 through the input/output interface 117 and processed based on the machine learning model 13.

In embodiments, the processor 111 is configured to perform one or more machine learning models 13, which can be implemented in hardware, software, firmware, or a combination thereof. The processor 111 may be, but is not limited to, a processing module, a Computer Processing Unit (CPU), an Application Processor (AP), a microcontroller, a digital signal processor. In embodiments, the processor 111 may include an image filter such as a high pass filter or a low pass filter to filter a specific factor in a fundus image. In addition, in embodiments, the processor 111 may communicate with a hardware controller such as the display adapter 119 to display a user interface on the display device 130. In embodiments, the processor 111 may access the memory unit 113 and execute commands stored in the memory unit 113 or one or more sequences of instructions to control the operation of the apparatus 100. The commands or sequences of instructions may be read in the memory unit 113 from computer-readable medium or media such as a static storage or a disk drive, but is not limited thereto. In alternative embodiments, a hard-wired circuitry which is equipped with a hardware in combination with software commands may be used. The hard-wired circuitry can replace the soft commands. The instructions may be an arbitrary medium for providing the commands to the processor 111 and may be loaded into the memory unit 113.

In embodiments, the system bus 112 may represent one or more of several possible types of bus structures, including a memory bus or memory controller, a peripheral bus, an accelerated graphics port, and a processor or local bus using any of a variety of bus architectures. For instance, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. In embodiments, the system bus 112, and all buses specified in this description can also be implemented over a wired or wireless network connection.

A transmission media including wires of the system bus 112 may include at least one of coaxial cables, copper wires, and optical fibers. For instance, the transmission media may take a form of sound waves or light waves generated during radio wave communication or infrared data communication.

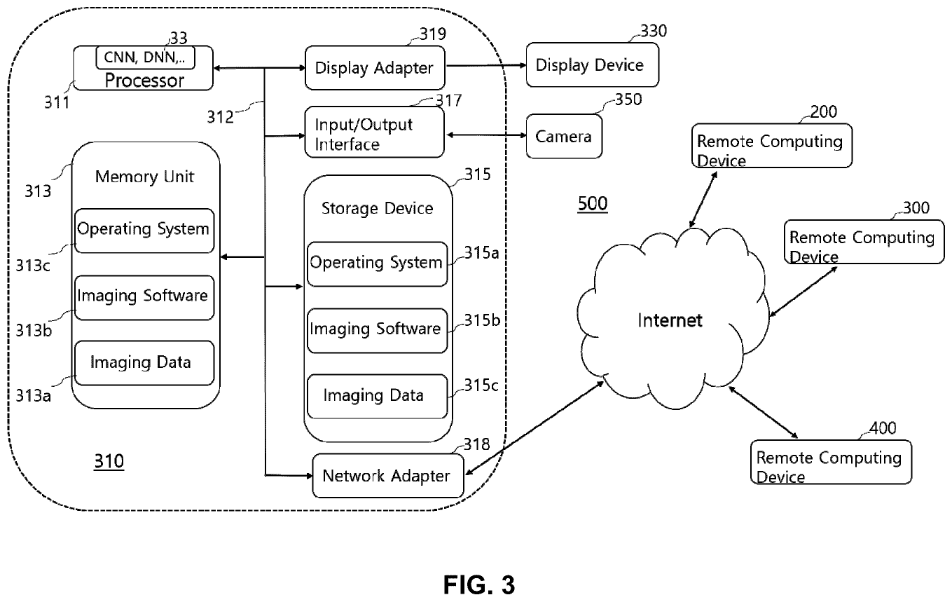

In embodiments, the apparatus 100 may transmit or receive the commands including messages, data, and one or more programs, i.e., a program code, through a network link or the network adapter 118. In embodiments, the network adapter 118 may include a separate or integrated antenna for enabling transmission and reception through the network link. The network adapter 118 may access a network and communicate with a remote computing devices 200, 300, 400 in FIG. 3.

In embodiments, the network may be, but is not limited to, at least one of LAN, WLAN, PSTN, and cellular phone networks. The network adapter 118 may include at least one of a network interface and a mobile communication module for accessing the network. In embodiments, the mobile communication module may be accessed to a mobile communication network for each generation such as 2G to 5G mobile communication network.

In embodiments, on receiving a program code, the program code may be executed by the processor 111 and may be stored in a disk drive of the memory unit 113 or in a non-volatile memory of a different type from the disk drive for executing the program code.

In embodiments, the computing device 110 may include a variety of computer-readable medium or media. The computer-readable medium or media may be any available medium or media that are accessible by the computing device 100. For example, the computer-readable medium or media may include, but is not limited to, both volatile and non-volatile media, removable or non-removable media.

In embodiments, the memory unit 113 may typically stores a database of fundus images that are used by the machine learning model 13, as described below in detail, although the database could be at some other location that is external to the remote computing devices 200, 300, 400 and accessible by the processor 111 via a network. The memory unit 113 may store a driver, an application program, data, and a database for operating the apparatus 100 therein. In addition, the memory unit 113 may include a computer-readable medium in a form of a volatile memory such as a random access memory (RAM), a non-volatile memory such as a read only memory (ROM), and a flash memory. For instance, it may be, but is not limited to, a hard disk drive, a solid state drive, an optical disk drive.

In embodiments, each of the memory unit 113 and the storage device 115 may be program modules such as the imaging software 113b, 115b and the operating systems 113c, 115c that can be immediately accessed so that a data such as the imaging data 113a, 115a is operated by the processor 111.

In embodiments, the machine learning model 13 may be trained to classify retinal features contained in fundus images and to predict, based on the classification, the presence of neurodevelopmental disorder disease in a human subject. The manner in which training may be performed and the manner in which the apparatus 100 is used to predict the presence of the neurodevelopmental disorder disease in a human subject are described below. Once trained, the machine learning model 13 may analyze fundus images captured by the image acquisition device like a camera 150 to identify and classify retinal features contained in the images. Based on the classification of the retinal features, the machine learning model 13 may predicts the presence of neurodevelopmental disorder disease such as autism spectrum disorder (ASD) and attention-deficit/hyperactivity disorder (ADHD) in human subject. In embodiments, the machine learning model 13 may be installed into at least one of the processor 111, the memory unit 113 and the storage device 115. The machine learning model 13 may use, but is not limited to, at least one of a deep neural network (DNN), a convolutional neural network (CNN) and a recurrent neural network (RNN), which are one of the machine learning algorithms.

FIG. 3 is a schematic diagram of an illustrative system for predicting neurodevelopmental disorder using a fundus image according to embodiments of the present disclosure.

As depicted, the system 500 may include a computing device 310 and one and more remote computing devices 200, 300, 400. In embodiments, the computing device 310 and the remote computing devices 200, 300, 400 may be connected to each other through a network. The components 310, 311, 312, 313, 315, 317, 318, 319, 330 of the system 500 are similar to their counterparts in FIG. 2. In embodiments, each of remote computing devices 200, 300, 400 may be similar to the apparatus 100 in FIG. 1. For instance, each of remote computing devices 200, 300, 400 may include each of the subsystems, including the processor 311, the memory unit 313, an operating system 313c, 315a, an imaging software 313b, 315b, an imaging data 313a, 315c, a network adapter 318, a storage device 315, an input/output interface 317 and a display adapter 319. Each of remote computing devices 200, 300, 400 may further include a display device 330 and a camera 350. In embodiments, the system bus 312 may connect the subsystems to each other.

In embodiments, the computing device 310 and the remote computing devices 200, 300, 400 may be configured to perform one or more of the methods, functions, and/or operations presented herein. Computing devices that implement at least one or more of the methods, functions, and/or operations described herein may comprise an application or applications operating on at least one computing device. The computing device may comprise one or more computers and one or more databases. The computing device may be a single device, a distributed device, a cloud-based computer, or a combination thereof.

It shall be noted that the present disclosure may be implemented in any instruction-execution/computing device or system capable of processing data, including, without limitation laptop computers, desktop computers, and servers. The present disclosure may also be implemented into other computing devices and systems. Furthermore, aspects of the present disclosure may be implemented in a wide variety of ways including software (including firmware), hardware, or combinations thereof. For example, the functions to practice various aspects of the present disclosure may be performed by components that are implemented in a wide variety of ways including discrete logic components, one or more application specific integrated circuits (ASICs), and/or program-controlled processors. It shall be noted that the manner in which these items are implemented is not critical to the present disclosure.

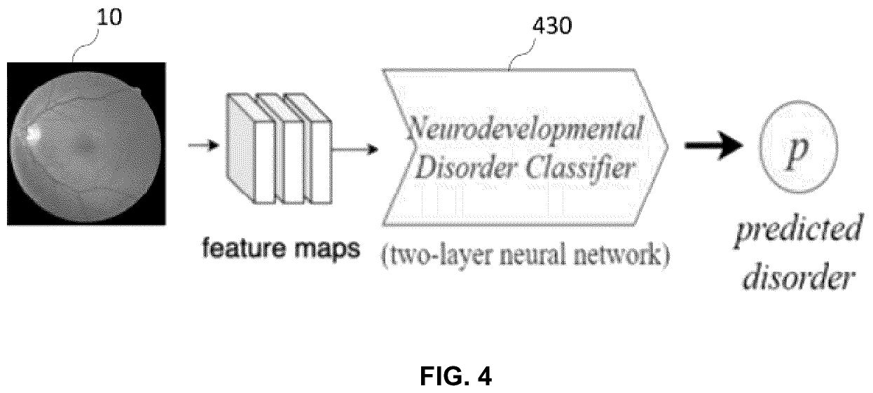

FIG. 4 is an exemplary diagram of a first architecture of a machine learning model for predicting of neurodevelopmental disorder according to embodiments of the present disclosure.

As depicted, a fundus image 10 is obtained from a subject and the feature maps may be generated by mapping an adversarial noise to the entire fundus image 10. Mapping the adversarial noise may be called by adversarial attack. The feature maps may be subsequently passed to the neurodevelopmental disorder classifier network 430, which may predict a presence of the neurodevelopmental disorder or a type of the neurodevelopmental disorder presented in the inputted feature maps. The presence of the neurodevelopmental disorder may be presented by numeric value. In this case, the adversarial noise is a factor that decreases the performance of the machine learning model as noise is added to the fundus image 10. This adversarial attack may advance the performance of the machine learning model 430 by training it with images that have noise added to it that will increase its loss function, creating an image that is much harder for the machine learning model 430 to classify. The machine learning model 430 may be a pre-trained convolutional neural network used in the previous learning stage in the FIG. 1.

By the way, in embodiments, to measure the error between the predicted disorder and its ground truth, the cross-entropy loss function may be utilized for a disorder and gender classifier.

$$Cross - entropy Loss Function ( loss of predicted disorder type ) \qquad Equation 1$$

$${L}_{cross - entropy - disorder} = - {log}_{e} P$$

$$Cross - entropy Loss Function ( loss of predicted gender ) \qquad Equation 2$$

$${L}_{cross - entropy - gender} = - {log}_{e} g$$

In the equations 1 and 2, P and g denote the probability of the predicted disorder type and gender, respectively.

For the architecture design, two-layer neural networks 430 may be utilized for both the gender classification and age regression networks, as well as the neurodevelopmental disorder classifier. Increasing the depth of the neural network beyond two layers did not significantly contribute to improving accuracy. Therefore, it is desirable that two layer neural networks are sufficient to achieve state-of-the-art performance.

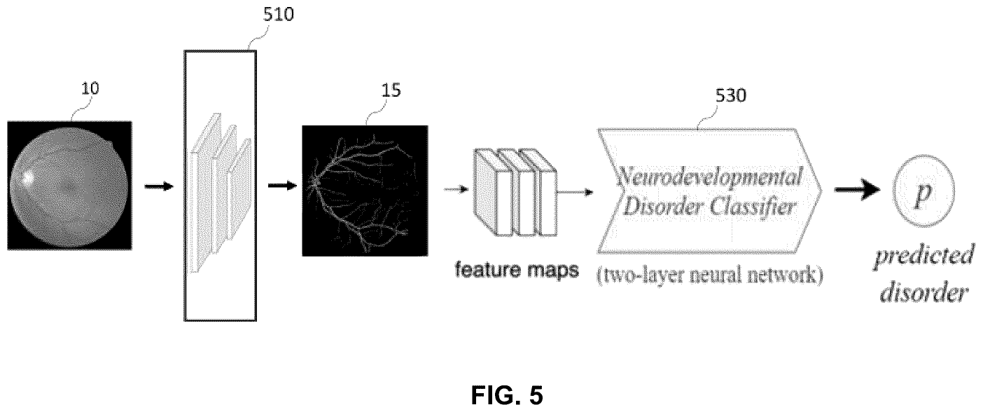

FIG. 5 is an exemplary diagram of a second architecture of a machine learning model for predicting of neurodevelopmental disorder according to embodiments of the present disclosure.

As depicted, a fundus image 10 obtained from a subject is input to a machine learning model 510. The machine learning model 510 may segment a region of interest (e.g., blood vessel) like biomarker in the fundus image 10 and may generate an image 15 with only the blood vessel isolated from the fundus image 10. The feature maps may generate by mapping an adversarial noise to the image 15 isolated from the fundus image 10. Mapping the adversarial noise may be called by adversarial attack. The feature maps may be subsequently passed to the neurodevelopmental disorder classifier network 530, which may predict a presence of the neurodevelopmental disorder or a type of the neurodevelopmental disorder presented in the inputted feature maps. The presence of the neurodevelopmental disorder may be presented by numeric value. In this case, the adversarial noise is a factor that decreases the performance of the machine learning model 530 as noise is added to the image 15. This adversarial attack may advance the performance of the machine learning model 530 by training it with images that have noise added to it that will increase its loss function, creating an image that is much harder for the machine learning model 530 to classify.

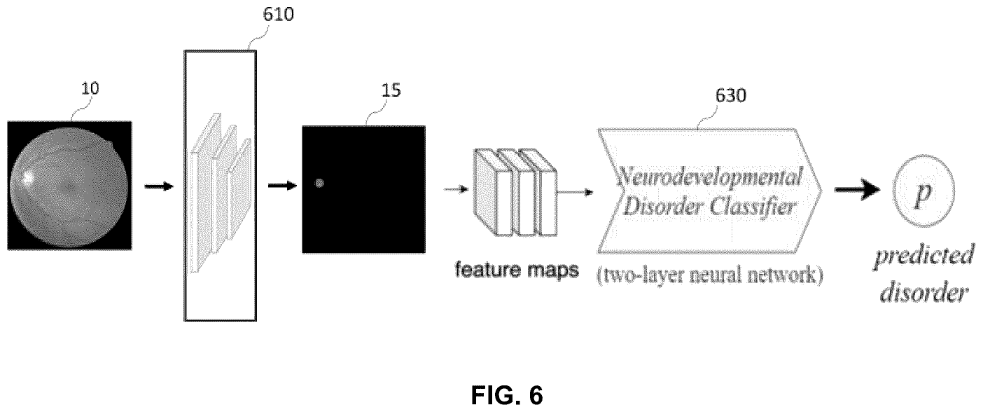

FIG. 6 is an exemplary diagram of a third architecture of a machine learning model for predicting of neurodevelopmental disorder according to embodiments of the present disclosure.

As depicted, a fundus image 10 obtained from a subject is input to a machine learning model 610. The machine learning model 610 may segment a region of interest (e.g., optic cup) like biomarker in the fundus image 10 and may generate an image 15 with only the optic cup isolated from the fundus image 10. The feature maps may generate by mapping an adversarial noise to the image 15 isolated from the fundus image 10. Mapping the adversarial noise may be called by adversarial attack. The feature maps may be subsequently passed to the neurodevelopmental disorder classifier network 630, which may predict a presence of the neurodevelopmental disorder or a type of the neurodevelopmental disorder presented in the inputted feature maps. The presence of the neurodevelopmental disorder may be presented by numeric value. In this case, the adversarial noise is a factor that decreases the performance of the machine learning model 630 as noise is added to the image 15. This adversarial attack may advance the performance of the machine learning model 630 by training it with images that have noise added to it that will increase its loss function, creating an image that is much harder for the machine learning model 630 to classify.

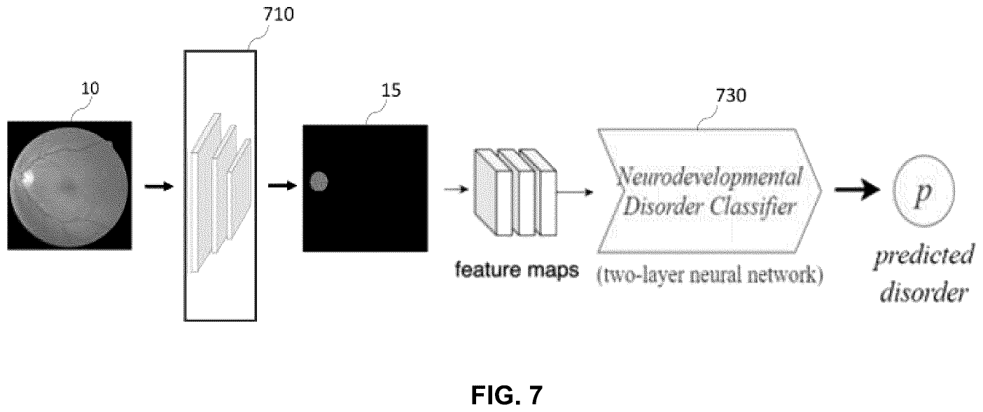

FIG. 7 is an exemplary diagram of a fourth architecture of a machine learning model for predicting of neurodevelopmental disorder according to embodiments of the present disclosure.

As depicted, a fundus image 10 obtained from a subject is input to a machine learning model 710. The machine learning model 710 may segment a region of interest (e.g., optic disc) like biomarker in the fundus image 10 and may generate an image 15 with only the optic cup isolated from the fundus image 10. The feature maps may generate by mapping an adversarial noise to the image 15 isolated from the fundus image 10. Mapping the adversarial noise may be called by adversarial attack. The feature maps may be subsequently passed to the neurodevelopmental disorder classifier network 730, which may predict a presence of the neurodevelopmental disorder or a type of the neurodevelopmental disorder presented in the inputted feature maps. The presence of the neurodevelopmental disorder may be presented by numeric value. In this case, the adversarial noise is a factor that decreases the performance of the machine learning model 730 as noise is added to the image 15. This adversarial attack may advance the performance of the machine learning model 730 by training it with images that have noise added to it that will increase its loss function, creating an image that is much harder for the machine learning model 730 to classify.

In embodiments, although the region of interest is represented by only blood vessel, optic cup, optic disc in the fundus images of FIGS. 5 to 7, the various other biomarkers included in the fundus image may be used as the region of interest.

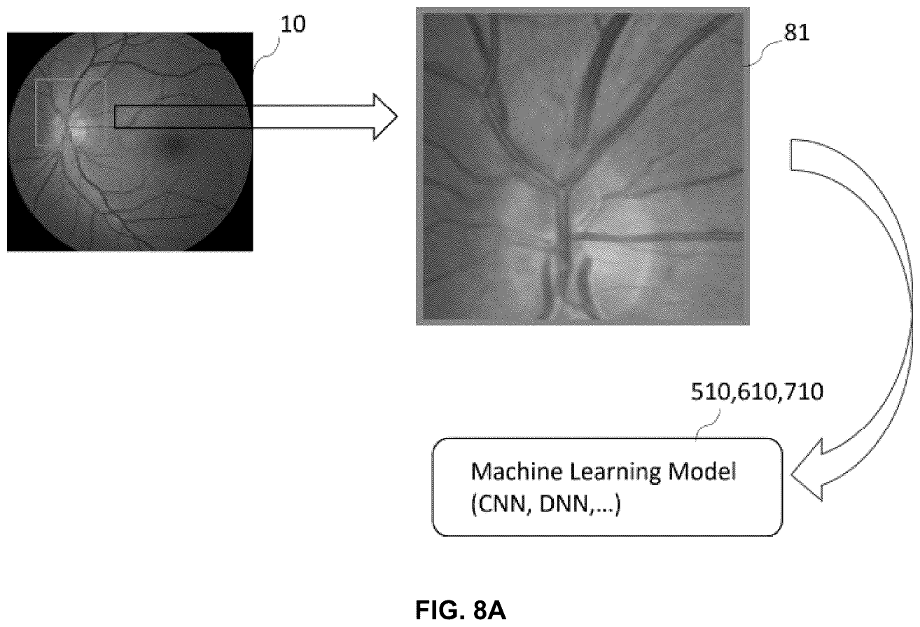

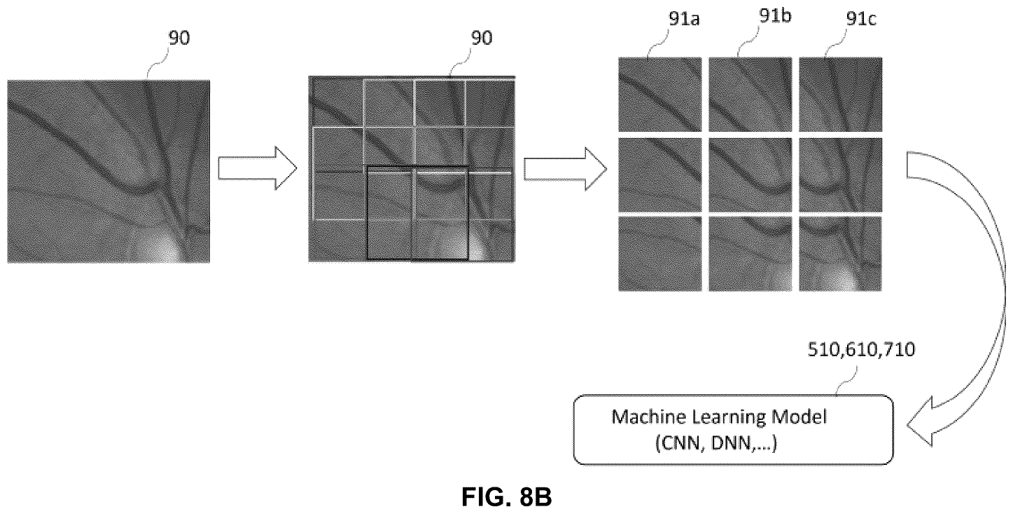

FIGS. 8A and 8B are a view showing an illustrative process for generating a pre-fundus image by apparatus according to embodiments of the present disclosure.

As depicted in FIG. 8A, in embodiments, initially an original fundus image 10 of the subject may be obtained by taking an image by a camera (not shown). After that, pre-fundus image 81 may be generated by pre-processing the original fundus image 10 by the processor 111 in FIG. 1 so that a specific region of the original fundus image is enlarged in order to more intensively check a feature information on the original fundus image 10. In this case, the specific region may be, but is not limited to, the optic nerve papilla region including retinal blood vessel and optic disc. The pre-fundus image 81 may be input to the machine learning model 510, 610, 710 of FIGS. 5 to 7.

As depicted in FIG. 8B, in embodiments, First, an enlarged fundus image 90 may be obtained by pre-processing an image like the original fundus image 10 described in FIG. 8A. The pre-processing may be executed by the processor 111 in FIG. 1. After that, the enlarged fundus image 90 may be partially partitioned by a plurality of areas. In this case, each of the plurality of areas may have the same size or different sizes. Each of the plurality of areas may overlap each other locally. A plurality of pre-fundus images 91a, 91b, 91c may be generated according to the partitioned areas. Finally, each of the plurality of pre-fundus images 91a, 91b, 91c may be input to the machine learning model 510, 610, 710 of FIGS. 5 to 7.

The pre-fundus image may be generated in various ways. For example, the pre-fundus image may be generated by rotating around a reference axis of the original fundus image, or/and the pre-fundus image may be generated by adjusting the contrast or brightness of the original fundus image or/and by flipping around a horizontal or vertical axis of the original fundus image. Thus, if the pre-fundus image inputs the machine learning model, thereby more improving prediction of the disease in the fundus image.

### Experiments

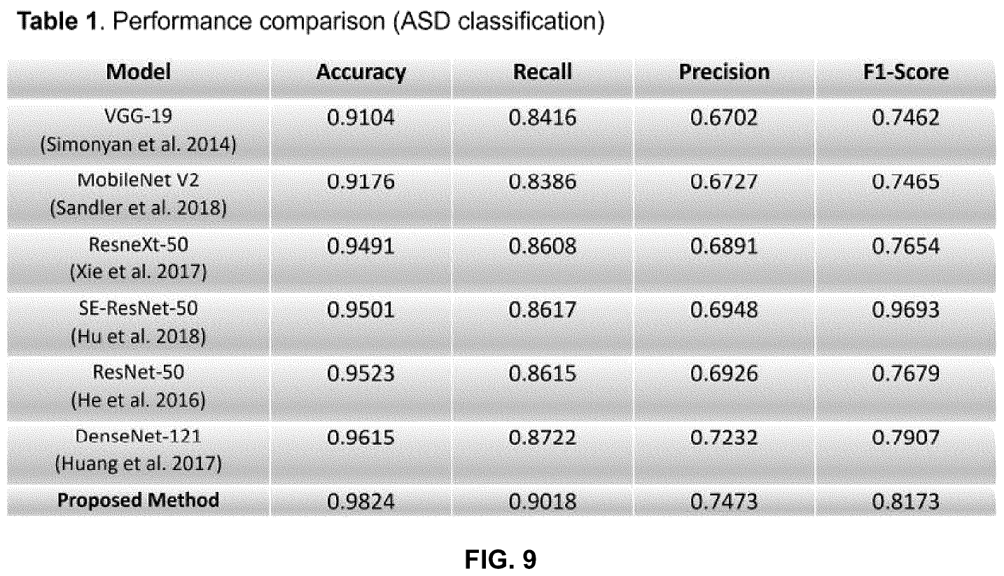

FIG. 9 shows a table of performance comparison of overall ASD classification results based on comparison with state-of the-art methods according to embodiments of the present disclosure.

The Table 1 shown in FIG. 9 lists the performance comparison of the proposed method vs. well-known several approaches for ASD classification. The well-known several approaches include architectures having networks such as VGG19, MobileNetV2, ResNext-50, SE-ResNET-50, ResNet-50, DenseNet-121. In Table 1, the accuracy, recall, precision, and FI-Score for 6 different convolutional neural networks and the proposed method. Looking at the figure, one can easily notice that the performance of the proposed method is much better compared to the other networks as it has significantly higher values of accuracy, recall, precision, and FI-Score. Because the accuracy is much higher than 91%, which is the cut off for whether or not the network could be used in real life diagnosis process, it could be used as an aid for psychiatrists in the diagnostic process of ASD although it is important to consider that the precision value is quite low, signifying that it may predict normal patients with ASD

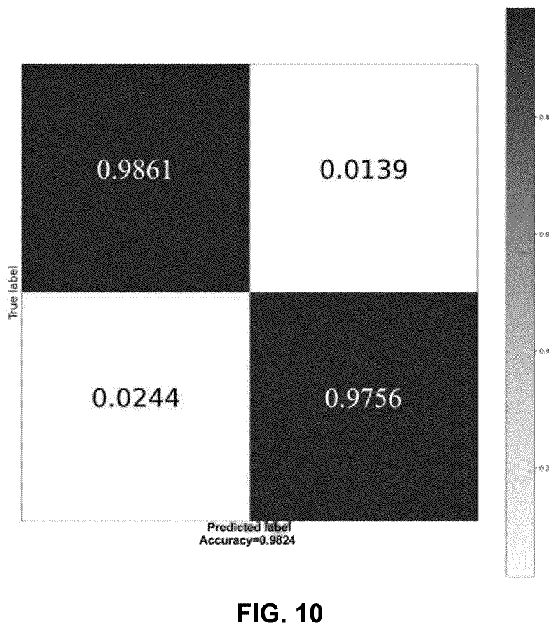

FIG. 10 shows a confusion matrix of a proposed method according to embodiments of the present disclosure.

Referring to FIG. 10, the confusion matrix shows the ratios of true positive, people who have ASD and are classified as ASD, to false positive, people who don't have ASD but are classified as ASD, and of true negative, people who don't have ASD and are classified as normal, to false negative, people who have ASD but are classified as normal. In the confusion matrix of the proposed approach, the values of true positive and negative are very close to 1.0, signifying how effective the proposed method is.

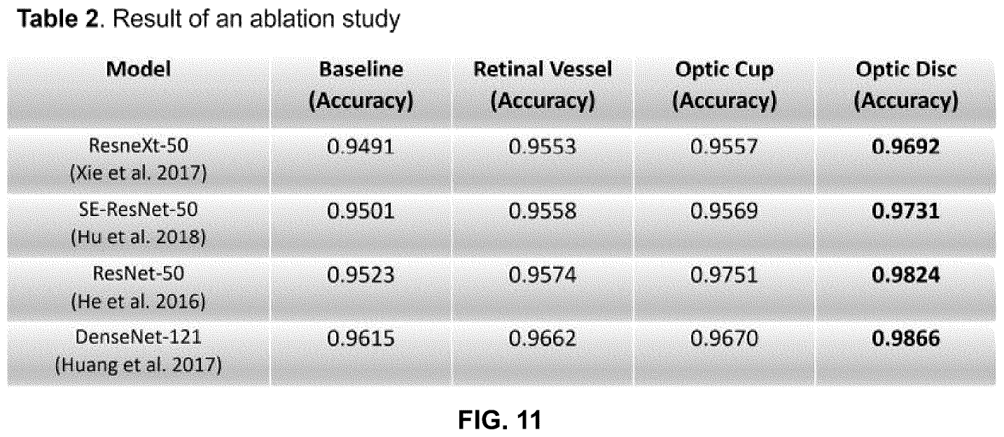

FIG. 11 shows a table of result of an ablation study based on various networks according to embodiments of the present disclosure.

Referring to FIG. 11, in Table 2, comparing the accuracy of each convolutional neural network to the baseline results, all of the networks trained with images that contain noise (Retina Vessel, Optic Cup, and Optic Disc) showed higher accuracy than the baseline network without any training for images that contain noise. This clearly shows that the accuracy has shown an increasing trend in the presence of the adversarial attack technique. Also, it is shown that the network trained with noise in the optic disc of the fundus images showed the highest accuracy among networks trained with noise in the retina vessel, optic cup, and optic disc. This indicates that the optic disc plays the most significant role in diagnosis of ASD using fundus images.

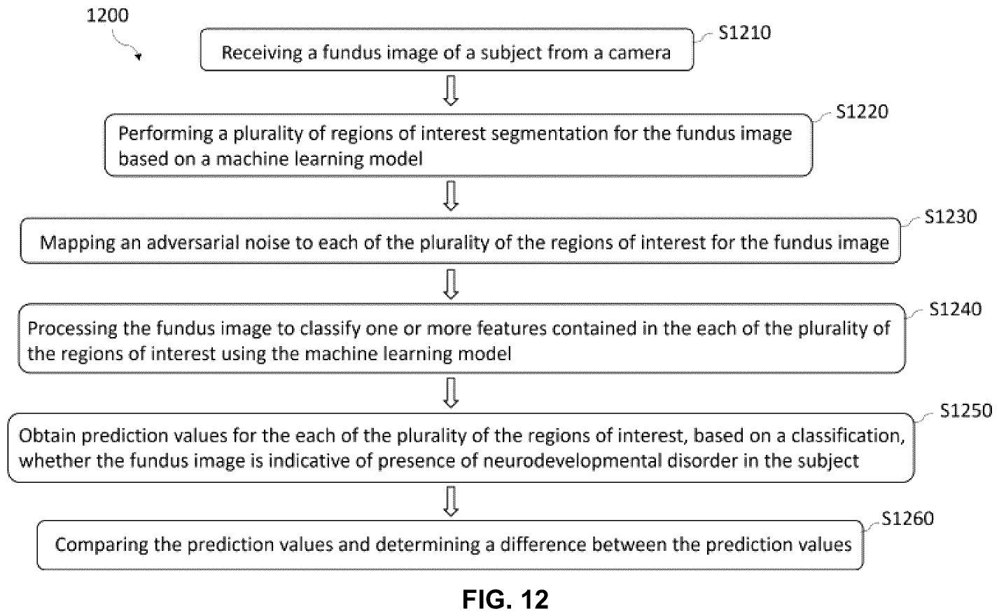

FIG. 12 is an exemplary flow diagram showing a prediction process of neurodevelopmental disorder using a fundus image according to embodiments of the present disclosure. The prediction process 1200 may be performed by a processor 111 installed into the computing device 110 in FIG. 2.

At step S1210, the processor 111 may receive a fundus image of human subject from an image acquisition device like a fundus camera. In embodiments, the fundus image may be stored in the memory unit 113, 313 or the storage device 115, 315 included to the computing device 110, 310 (as discussed in conjunction with FIGS. 1 and 2). At step S1220, the processor 111 may segment a plurality of regions of interest for the fundus image based on a machine learning model. In embodiments, the plurality of regions of interest may include at least one of a retinal vessel, optic cup, optic disc as a biomarker in the fundus image. At step S1230, the processor 111 may map an adversarial noise to each of the plurality of the regions of interest for the fundus image. In embodiments, the adversarial noise may have at least one of gradation levels of R, G, and B pixels of the fundus image, a color of R, G, and B pixels of the fundus image, and a contrast ratio of the fundus image. Also, each of the adversarial noise mapping to each of biomarkers (e.g., retinal vessel, optic cup, optic disc) may have a same value. At step S1240, the processor 111 may process the fundus image to classify one or more features contained in the each of the plurality of the regions of interest. As the biometric information of the fundus image, the features or the feature information may be classified by a machine learning model 13, 33 installed into the processor 111. In embodiments, the feature information may be stored in the memory unit 113, 313 or the storage device 115, 315. At step S1250, the processor 111 may obtain prediction values for the each of the plurality of the regions of interest, based on a classification, whether the fundus image is indicative of presence of neurodevelopmental disorder such as autism spectrum disorder (ASD) and attention-deficit/hyperactivity disorder (ADHD) in a human subject. In embodiments, the prediction values may be represented by numerical value. At step S1260, the processor 111 may compare the prediction values of the neurodevelopmental disorder and determine a difference between the prediction values. In embodiment, the difference may be displayed on the display device 130.

Embodiments of the present disclosure may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present disclosure may further relate to computer products with a non-transitory, tangible computer-readable medium that have computer code thereon for performing various computer-implemented operations. The media and computer code may be those specially designed and constructed for the purposes of the present disclosure, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present disclosure may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present disclosure. One skilled in the art will also recognize that a number of the elements described above may be physically and/or functionally separated into sub-modules or combined together.

It will be appreciated to those skilled in the art that the preceding examples and embodiment are exemplary and not limiting to the scope of the present invention. It is intended that all permutations, enhancements, equivalents, combinations, and improvements thereto that are apparent to those skilled in the art upon a reading of the specification and a study of the drawings are included within the true spirit and scope of the present invention.

What is claimed is:

1. An apparatus for enhancing prediction of neurodevelopmental disorder using a fundus image of a subject, comprising:
a processor; and
a memory comprising one or more sequences of instructions which, when executed by the processor, causes steps to be performed comprising:
receiving the fundus image;
segmenting a region of interest for the fundus image based on a machine learning model;
mapping an adversarial noise to the region of interest;
processing the fundus image to classify one or more features contained in the region of interest, which is mapped by the adversarial noise, using the machine learning model; and
predicting, based on a classification, whether the fundus image is indicative of presence of neurodevelopmental disorder in the subject.

2. The apparatus of claim 1, wherein the region of interest includes at least one of a retinal vessel, optic cup, optic disc in the fundus image.

3. The apparatus of claim 1, wherein the adversarial noise includes at least one of gradation levels of R, G, and B pixels of the fundus image, a color of R, G, and B pixels of the fundus image, and a contrast ratio of the fundus image.

4. The apparatus of claim 1, wherein the neurodevelopmental disorder includes autism spectrum disorder or attention-deficit/hyperactivity disorder.

5. The apparatus of claim 1, wherein the steps further comprises:
generating images in which a specific area of the fundus image is enlarged, after receiving the fundus image.

6. An apparatus for enhancing prediction of neurodevelopmental disorder using a fundus image of a subject, comprising:
a processor; and
a memory comprising one or more sequences of instructions which, when executed by the processor, causes steps to be performed comprising:
receiving the fundus image;
segmenting a plurality of regions of interest for the fundus image based on a machine learning model;
mapping an adversarial noise to each of the plurality of the regions of interest;
processing the fundus image to classify one or more features contained in the each of the plurality of the regions of interest, which is mapped by the adversarial noise, using the machine learning model;
obtaining prediction values for the each of the plurality of the regions of interest, based on a classification, whether the fundus image is indicative of presence of neurodevelopmental disorder in the subject; and
comparing the prediction values and determining a difference between the prediction values.

7. The apparatus of claim 6, wherein the adversarial noise mapping to each of the plurality of the regions of interest has a same value.

8. The apparatus of claim 6, wherein the region of interest includes at least one of a retinal vessel, optic cup, optic disc in the fundus image.

9. The apparatus of claim 6, wherein the adversarial noise includes at least one of gradation levels of R, G, and B pixels of the fundus image, a color of R, G, and B pixels of the fundus image, and a contrast ratio of the fundus image.

10. A non-transitory computer-readable medium or media comprising one or more sequences of instructions which, when executed by a processor, causes steps for predicting of neurodevelopmental disorder using a fundus image of a subject, comprising:
receiving the fundus image;
segmenting a plurality of regions of interest for the fundus image based on a machine learning model;
mapping an adversarial noise to each of the plurality of the regions of interest;
processing the fundus image to classify one or more features contained in the each of the plurality of the regions of interest, which is mapped by the adversarial noise, using the machine learning model; and
obtaining prediction values for the each of the plurality of the regions of interest, based on a classification, whether the fundus image is indicative of presence of neurodevelopmental disorder in the subject; and
comparing the prediction values and determining a difference between the prediction values.

11. The non-transitory computer-readable medium or media of claim 10, wherein the region of interest includes at least one of a retinal vessel, optic cup, optic disc in the fundus image.

12. The non-transitory computer-readable medium or media of claim 10, wherein the adversarial noise includes at least one of gradation levels of R, G, and B pixels of the fundus image, a color of R, G, and B pixels of the fundus image, and a contrast ratio of the fundus image.

13. The non-transitory computer-readable medium or media of claim 10, wherein the adversarial noise mapping to each of the plurality of the regions of interest has a same value.

14. The non-transitory computer-readable medium or media of claim 10, wherein the neurodevelopmental disorder includes autism spectrum disorder or attention-deficit/hyperactivity disorder.
